(19) United States
(12) Patent Application Publication
(10) Pub. No.: US 2007/0051369 A1
(43) Pub. Date: Mar. 8, 2007

(54) Apparatus, method, and medium calculating calorie consumption

(75) Inventors: Ji Hyun Choi, Seoul (KR); Jin Sang Hwang, Suwon-si (KR); Kun Soo Shin, Seongnam-si (KR)

Correspondence Address: STAAS & HALSEY LLP, SUITE 700, 1201 NEW YORK AVENUE, N.W., WASHINGTON, DC, 20005, US

(73) Assignee: SAMSUNG ELECTRONICS CO., LTD., Suwon-si (KR)

(21) Appl. No.: 11/417,176

(22) Filed: May 4, 2006

(30) Foreign Application Priority Data
Sep. 8, 2005 (KR) 10-2005-0083842

(51) Int. Cl. A61M 16/00; A62B 7/00

(52) U.S. Cl. 128/204.21

(57) ABSTRACT

An apparatus, medium, and method for calculating calorie consumption of a user performing physical activity. More particularly, an apparatus, medium, and method for calculating calorie consumption can determine a user's activity pattern via an acceleration sensor attached to the user, and calculate the user's calorie consumption by using a predetermined slope value according to the activity pattern and a heart rate measured via a heart rate sensor attached to the user. Accordingly it is possible to calculate calorie consumption of a user performing physical activity by considering the user's heart rate and the type of physical activity and accurately calculate the calorie consumption by using both an acceleration sensor and a heart rate sensor.

### CROSS-REFERENCE TO RELATED APPLICATIONS

This application claims the benefit of Korean Patent Application No. 10-2005-0083842, filed Sep. 8, 2005, in the Korean Intellectual Property Office, the disclosure of which is incorporated herein by reference.

### BACKGROUND OF THE INVENTION

1. Field of the Invention

Embodiments of the present invention relate to an apparatus, method, and medium calculating calorie consumption of a user performing some type of physical activity. More particularly, embodiments of the present invention relate to an apparatus, method, and medium calculating calorie consumption by determining a user's activity pattern via an acceleration sensor attached to the user, and calculating the user's calorie consumption by using a predetermined slope value based on the determined activity pattern and a heart rate measured via a heart rate sensor attached to the user.

2. Description of the Related Art

We define Ubiquitous to mean an information communication environment where a user can be free to access networks, network devices, and/or computers at any place without having to be conscious of the surrounding networks, network devices, or computers, for example. If such a Ubiquitous is commercialized, anyone would readily be able to use information technology not only in their homes or cars, but most anywhere, e.g., even on a mountaintop. Also, the commercialization of Ubiquitous may expand the information technology industry or the scope corresponding thereto by increasing the number of computer users who are connected to networks. Because of the advantages of Ubiquitous, with users being able to access networks without restriction to time and place, as well as having greater portability and convenience, countries worldwide are currently expanding development and competing in Ubiquitous-related technologies.

Ubiquitous-related technology may be applied to any field in human life. In particular, Ubiquitous-HealthCare (hereinafter, U-HealthCare) has recently been in the spotlight as a notable technology area, e.g., due to the recent health craze and “well-being” boom. U-HealthCare Ubiquitous technology enables anyone to readily receive medical services at any time and at any place by installing medical service-related chips or sensors in places of the user's living space, for example. With U-HealthCare, various types of medical attention, such as physical examinations, disease management, emergency care, consultation with a doctor and the like, which currently may only be performed in hospitals, may be naturally integrated into our daily lives, and thus may be accomplished without going to a hospital.

As an example of U-Healthcare, portable calorie calculators have currently been in the spotlight because of concerns about an individual's diet practices, noting that many people may have tried modifying their diets at least once. Correspondingly, calorie consumption has been generally calculated only in hospitals or clinic centers. However, a user can easily calculate calorie consumption without restriction to time or place via portable calorie calculators, which have resulted in more effective diet regiments.

Conventional calorie consumption calculators may be classified as falling within one of an accelerometer method, a heart rate method, and a heart rate-accelerometer method.

According to one conventional calorie consumption calculator, of the accelerometer method type, in the case of using a single acceleration sensor, if a user is weight training or cycling so that a sensor attached to the user does not move, the acceleration sensor may not detect the motion of the user. Thus, the acceleration sensor may not properly calculate calorie consumption. Also, when multiple acceleration sensors are used to solve the above problems, additional costs are incurred to connect the sensors and more electric power is required. Thus, overall, the benefits available from the multiple acceleration sensors may, at least, be countered by the increased costs and power requirements.

According to one conventional calorie consumption calculator, of the heart rate method type, calorie consumption may be calculated simply by measuring a heart rate. Thus, any correlation between types of physical activity and calorie consumption would not be considered and a user is further required to manually input each differing type of physical activity. Also, for the same type of physical activity basic heart rates may be different for each user. Thus, here, calorie consumption may not be accurately calculated.

To solve the aforementioned problems, a calorie consumption calculator, of the heart rate-accelerometer method type has been developed to measure heart rate and acceleration simultaneously. However, the conventional calorie consumption calculators estimate calorie consumption by determining only whether a user performs physical activity and by taking a user's temperature, emission, and impedance. In this instance, the calorie consumption calculator does not perform any calibration for each individual user. Accordingly, according to such conventional systems, the physical characteristic of each user is not reflected. Also, the characteristic of calorie consumption, according to each separate type of physical activity is not considered. Thus, an accurate calculation of calorie consumption, according to the separate types of physical activity and the individual characteristics of each user, is not expected.

As an example of such conventional systems, Japanese Patent Publication No. 10-17560 sets forth a “Calorie meter” having a configuration of calculating calorie consumption by using an accelerometer and a heart rate sensor. Here, the physical activity being performed by a user is determined by an acceleration sensor, and then the calorie meter calculates, based on the acceleration sensor, the user's calorie consumption. When it is determined that the user is not performing a physical activity, the calorie meter calculates the user's calorie consumption based on the heart rate sensor. However, in the case of calculating a user's calorie consumption based on an acceleration sensor, calories may be calculated differently in accordance with the type of physical activity and position of the attached acceleration sensor. Namely, the accuracy of the calorie calculation is not guaranteed.

Also, Korean Patent Publication No. 2002-059835 sets forth a “Calorie calculator” which detects a user's motion and calculates a quantity of motion by using an acceleration sensor, and measures the user's heart rate via a heart rate sensor. After this, the calorie calculator calculates the total daily calorie consumption of the user by using the quantity of motion and heart rate. However, this system calculates calorie consumption by using only the quantity of motion and heart rate of a user, and does not consider any calorie consumption which may differ depending on the type of physical activity being performed. Thus, again, calorie consumption may not be calculated accurately.

Because of the aforementioned problems in the conventional systems, there is a need for the development of an apparatus, method, and medium to calculate calorie consumption, with calibration based on the characteristics of each user, and thereby generate a more accurate calorie consumption calculation by using an activity pattern of physical activity a user performs and the heart rate of the user, and potentially automatically transmitting the calculated calorie consumption to an external server or terminal.

### SUMMARY OF THE INVENTION

To solve the aforementioned conventional problems, embodiments of the present invention include a calorie consumption calculating apparatus, medium, and method capable of calculating calorie consumption of a user performing physical activity by considering the user's heart rate and the type of the physical activity, and calculating an accurate calorie consumption by using both an acceleration sensor and a heart rate sensor.

Embodiments of the present invention also include a calorie consumption calculating apparatus, medium, and method capable of measuring a user's normal heart rate and calorie consumption and reflecting the measured normal heart rate on the calculation of calorie consumption, and thereby, for each user, calculating substantially accurate calorie consumption, which may vary according to the physical characteristics of each user.

Embodiments of the present invention also include a calorie consumption calculating apparatus, capable of determining a user's activity pattern by measuring the user's heart rate via a heart rate sensor when the user's activity pattern is not detected by an acceleration sensor, and calculating accurate calorie consumption of the user according to an activity pattern, which may be hard to detect via an acceleration sensor, such as cycling or weight training.

Embodiments of the present invention also include a calorie consumption calculating apparatus, medium, and method, which are suitable for embodying U-HealthCare by transmitting and displaying a user's calculated calorie consumption to an external communication terminal via wired/wireless communication.

To achieve the above and/or other aspects and advantage, embodiments of the present invention include a method for calculating calorie consumption of a body, including detecting whether the body is in motion based on an acceleration sensor for the body, and determining an activity pattern of the body according to a detected motion, if the motion is detected, measuring the body's heart rate based on a heart rate sensor for the body, and calculating the body's calorie consumption based on a predetermined relationship, between heart rates and the determined activity pattern, and the measured heart rate.

The predetermined relationship may be a slope value representing an identifiable change in heart rates for a particular activity pattern.

The method may further include maintaining an activity pattern table recording at least one activity pattern identifier and a corresponding predetermined relationship, between heart rates and a respective activity pattern identifier, for activity pattern identifiers in the activity pattern table.

In addition, the method may include measuring the body's normal heart rate and normal calorie consumption, the normal heart rate and calorie consumption being measured when the body does not perform a predetermined physical activity, wherein the calculating of the body's calorie consumption by using the predetermined relationship and the measured heart rate includes multiplying the predetermined relationship by a subtraction of the normal heart rate from the measured heart rate and then adding the normal calorie consumption.

The measuring of the body's normal calorie consumption may include when a motion according to a first activity pattern is detected for the body, extracting a first predetermined relationship corresponding to the first activity pattern from an activity pattern table, measuring the body's first heart rate according to the first activity pattern, calculating the body's first calorie consumption according to the first activity pattern by using an acceleration signal output from the acceleration sensor, and multiplying the first predetermined relationship by a subtraction of the normal heart rate from the first heart rate, and subtracting a result of the multiplication of the first predetermined relationship from the first calorie consumption.

The method may include measuring the body's heart rate via the heart rate sensor and calculating the calorie consumption during a physically motionless period, based on the body not being detected to be in motion by the acceleration sensor, based on the measured body's heart rate, amplifying the body's acceleration signal, measured from the acceleration sensor, when the measured heart rate is more than a predetermined value, and detecting the body's activity pattern based on the amplified acceleration signal.

Still further, the method may include transmitting the calculated calorie consumption to a predetermined communication terminal through a wired/wireless network, wherein the communication terminal receives the calculated calorie consumption and displays the calculated calorie consumption on a predetermined display or the communication terminal receives the activity pattern and calculated calorie consumption with time data and displays the activity pattern and calculated calorie consumption with time data on a predetermined display.

The method may also include calibrating potential predetermined relationships for corresponding activity patterns to reflect physical characteristics of the body by requiring the body to perform a predetermined testing process.

To achieve the above and/or other aspects and advantage, embodiments of the present invention include an apparatus, including an acceleration sensor for a body to detect motion of the body and to output an acceleration signal relative to a detected motion, an activity pattern determination unit to determine the body's activity pattern based on the acceleration signal and a predetermined relationship, between heart rates and the determined activity pattern, corresponding to the determined activity pattern, a heart rate sensor for the body to measure the body's heart rate, and a data controller to calculate the body's calorie consumption based on the predetermined relationship and the measured heart rate.

The predetermined relationship may be a slope value representing an identifiable change in heart rates for a particular activity pattern.

The apparatus may further include a memory storing at least one activity pattern identifier and a corresponding predetermined relationship, between heart rates and a respective activity pattern identifier, for activity pattern identifiers in the activity pattern table.

The heart rate sensor may further measure the body's normal heart rate, the normal heart rate being measured when the body does not perform a predetermined physical activity, and the data controller may further calculate the body's normal calorie consumption by using an acceleration signal of the acceleration sensor, the normal calorie consumption being measured when the body does not perform the predetermined physical activity, and calculate calorie consumption by multiplying the predetermined relationship by a subtraction of the normal heart rate from the measured heart rate and then adding the normal calorie consumption.

When a motion of the body for a first activity pattern is detected via the acceleration sensor, the activity pattern determination unit may extract a corresponding first predetermined relationship corresponding to the first activity pattern from an activity pattern table, the heart rate sensor may measure the body's first heart rate according to the first activity pattern, and the data controller may calculate the body's first calorie consumption according to the first activity pattern based on the acceleration signal output from the acceleration sensor, and calculate the body's normal calorie consumption by multiplying the first predetermined relationship by a subtraction of the normal heart rate from the first heart rate, and subtracting a result of the multiplication of the first predetermined relationship from the first calorie consumption.

The apparatus may include a communication module to transmit the calculated calorie consumption to an external communication terminal via a predetermined wired/wireless network.

To achieve the above and/or other aspects and advantage, embodiments of the present invention include a calorie consumption system, including an acceleration sensor for a body to detect motion of the body and to output an acceleration signal relative to a detected motion, an activity pattern determination unit to determine the body's activity pattern based on the acceleration signal and a predetermined relationship, between heart rates and the determined activity pattern, corresponding to the determined activity pattern, a heart rate sensor for the body to measure the body's heart rate, and a data controller to calculate the body's calorie consumption based on the predetermined relationship and the measured heart rate, wherein at least the acceleration sensor and heart rate sensor are connected with the body.

The activity pattern determination unit and the data controller may be separated from the body and the acceleration sensor and heart rate sensor.

In addition, the predetermined relationship may be a slope value representing an identifiable change in heart rates for a particular activity pattern.

The system may include a memory storing at least one activity pattern identifier and a corresponding predetermined relationship, between heart rates and a respective activity pattern identifier, for activity pattern identifiers in the activity pattern table.

The heart rate sensor may further measure the body's normal heart rate, the normal heart rate being measured when the body does not perform a predetermined physical activity, and the data controller may further calculate the body's normal calorie consumption by using an acceleration signal of the acceleration sensor, the normal calorie consumption being measured when the body does not perform the predetermined physical activity, and calculate calorie consumption by multiplying the predetermined relationship by a subtraction of the normal heart rate from the measured heart rate and then adding the normal calorie consumption.

When a motion of the body for a first activity pattern is detected via the acceleration sensor, the activity pattern determination unit may extract a corresponding first predetermined relationship corresponding to the first activity pattern from an activity pattern table, the heart rate sensor may measure the body's first heart rate according to the first activity pattern, and the data controller may calculate the body's first calorie consumption according to the first activity pattern based on the acceleration signal output from the acceleration sensor, and calculate the body's normal calorie consumption by multiplying the first predetermined relationship by a subtraction of the normal heart rate from the first heart rate, and subtracting a result of the multiplication of the first predetermined relationship from the first calorie consumption.

The system may further include a communication module to transmit the calculated calorie consumption to an external communication terminal via a predetermined wired/wireless network.

To achieve the above and/or other aspects and advantage, embodiments of the present invention may include at least one medium including computer readable code to implement a method embodiment of the present invention.

Additional aspects and/or advantages of the invention will be set forth in part in the description which follows and, in part, will be apparent from the description, or may be learned by practice of the invention.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIG. 1 illustrates a calorie consumption calculating apparatus, according to an embodiment of the present invention;

FIG. 2 illustrates graphs showing correlations between heart rate and calorie consumption, according to an embodiment of the present invention;

FIG. 3 illustrates an activity pattern table recorded in a memory, according to an embodiment of the present invention;

FIG. 4 illustrates a correlation between the quantity of motion and calorie consumption, according to an embodiment of the present invention;

FIG. 5 illustrates a calorie consumption calculating method, according to an embodiment of the present invention;

FIG. 6 illustrates a calibration method based on the characteristics of each user by a calorie consumption calculating apparatus, according to an embodiment of the present invention; and

FIG. 7 illustrates a calorie consumption calculating method for when an acceleration sensor of a calorie consumption calculating apparatus does not detect a user's motion, according to an embodiment of the present invention.

### DETAILED DESCRIPTION OF THE PREFERRED EMBODIMENTS

Reference will now be made in detail to embodiments of the present invention, examples of which are illustrated in the accompanying drawings, wherein like reference numerals refer to the like elements throughout. Embodiments are described below to explain the present invention by referring to the figures.

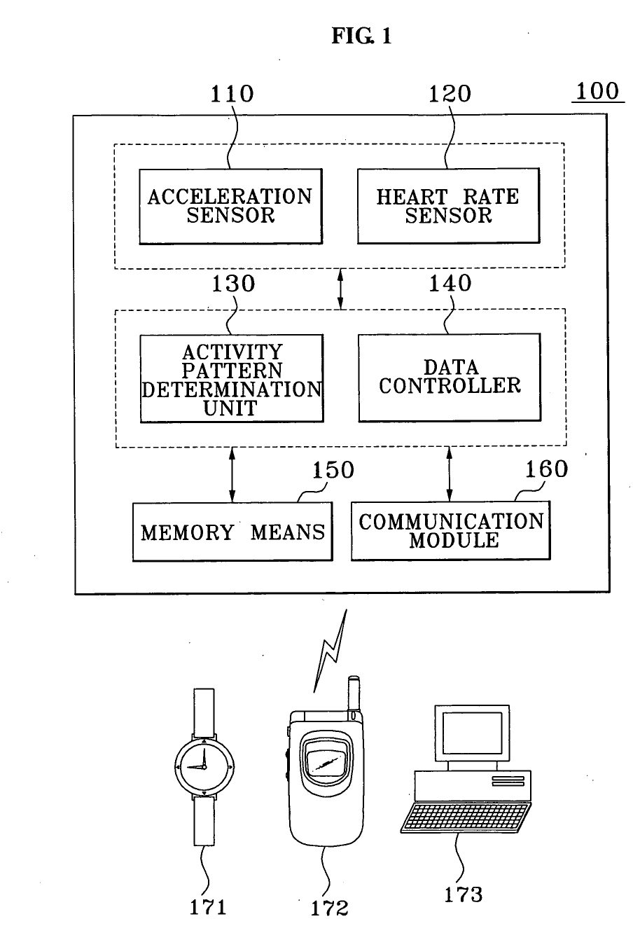

FIG. 1 illustrates a calorie consumption calculating apparatus, according to an embodiment of the present invention.

The calorie consumption calculating apparatus may include a sensor portion (110, 120), a control portion (130, 140), a memory 150, and a communication module 160. The sensor portion may include an acceleration sensor 110 and a heart rate sensor 120, for example. The control portion may include an activity pattern determination unit 130, and a data controller 140, for further example. In this instance, the sensor portion, the control portion, the memory 150, and the communication module 160 may be embodied in one apparatus, e.g., to be attached to the body of a user. Alternatively, only the sensor portion may be attached to the body of a user, and the control portion, the memory 150 and the communication module 160 may be embodied in a separate device from the sensor portion, e.g., not attached to the body of the user, noting that alternative embodiments are equally available.

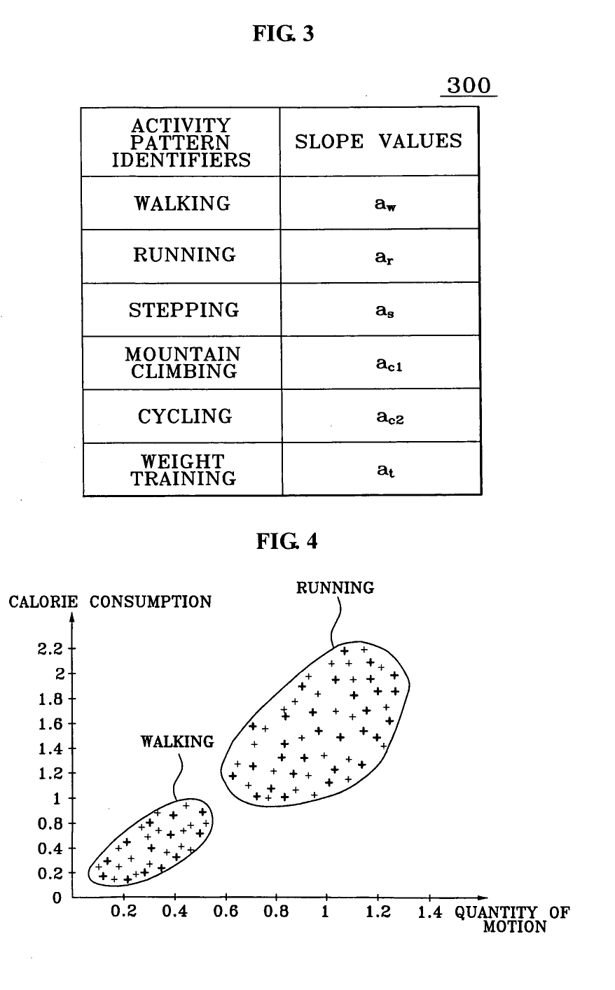

The memory 150 may maintain an activity pattern table, storing/recording at least one activity pattern identifier and a corresponding predetermined relationship, e.g., a slope value, with each activity pattern identifier. The activity pattern table will be described in greater detail below with reference to FIG. 3.

The acceleration sensor 110 may be attached to the body of a predetermined user, detect the user's motion, and output an acceleration signal according to the motion. To operate as described above, the acceleration sensor 110 may include an accelerometer, a fluxgate sensor, and a gyroscope, for example, noting that alternative embodiments are equally available. The accelerometer may generate an acceleration signal with respect to vibrations in the direction of three axes of X, Y, and Z according to the user's motion, for example. Also, the acceleration sensor 110 may further generate an external acceleration signal such as acceleration against gravity or the like, for example.

The heart rate sensor 120 may be attached to the body of a user and measure a user's heart rate. To measure the heart rate, the heart rate sensor 120 may include at least one piezoelectric sensor, photoelectric sensor or the like, which is generally used to measure a pulse or heart rate, for example.

The activity pattern determination unit 130 may determine the user's activity pattern by reading the acceleration signal and extracting a corresponding predetermined relationship, e.g., a slope value, with the determined activity pattern from the activity pattern table of the memory 150. To explain how the activity pattern determination unit 130 determines an activity pattern, the principle of a calorie consumption calculating method, according to a correlation between heart rate and calorie consumption, will now be briefly explained in greater detail.

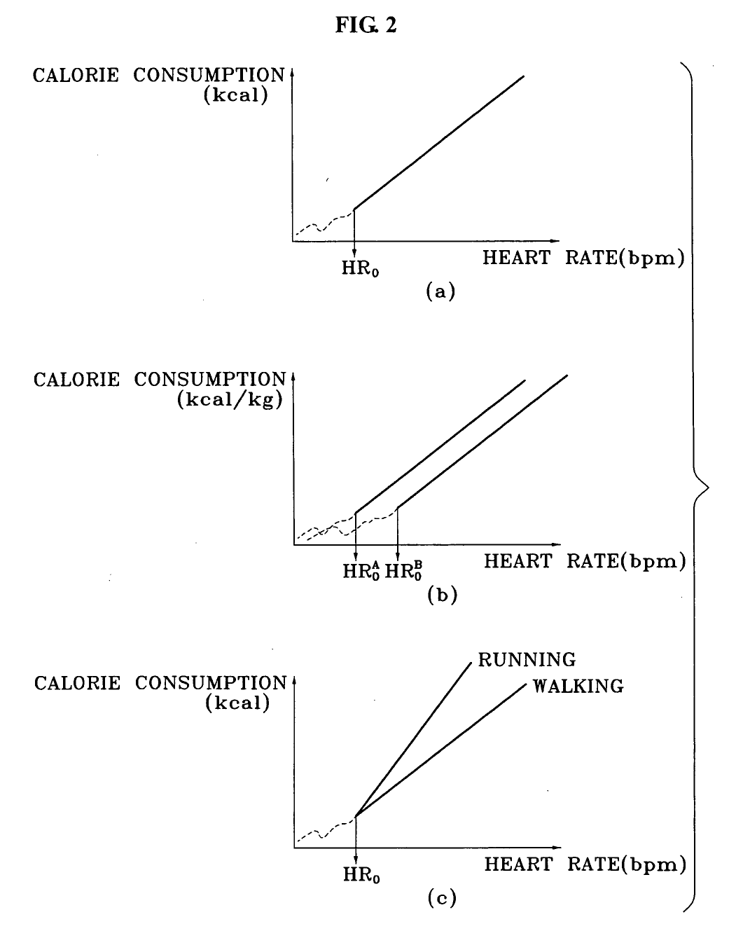

FIG. 2 illustrates graphs showing the correlation between heart rate and calorie consumption, according to an embodiment of the present invention.

A person's heart rate and calorie consumption may be in proportion to each other. In other words, the higher the intensity of physical activity, the higher the heart rate. Accordingly, more calories will be consumed. As illustrated in illustration (a) of FIG. 2, as the heart rate increases, calorie consumption also increases. Accordingly, the heart rate and calorie consumption may be indicated to have a linear relation, for example, in a graph. However, this may be applicable only to the case when a user performs certain types of physical activity. When a user does not perform physical activity, the relationship between the heart rate and calorie consumption may be defined based of the physical characteristic of each user.

As illustrated, HR₀ indicated on the heart rate axis in illustration (a) of FIG. 2, would be a user's normal heart rate. Accordingly, it may be determined that the user is performing some physical activity where the heart rate is more than HR₀. Also, when the heart rate is less than HR₀, it may be determined that the user is in a normal state where the user does not perform physical activity. Accordingly, the heart rate HR₀ may be equal to a maximum heart rate when the user does not perform physical activity or, as another example, may be an average of previously measured heart rates when the user does not perform physical activity.

The calorie consumption may be different depending on the normal heart rate of each user. Namely, the heart rate HR₀ may be measured differently depending on the physical characteristic of each user. Accordingly, even when users have the same heart rate, while performing some physical activity, calorie consumption of each user may be different. As shown in illustration (b) of FIG. 2, the normal heart rate of user A may be HR^{A}₀ and the normal heart rate of user B may be HR^{B}₀. As described above, the normal heart rates for each user may be different depending on the physical characteristics of each user. Accordingly, the normal heart rate of each user may be an important factor to calculate calorie consumption.

Further, an additional important factor is that the increased speed of the heart rate may be different depending on the type of physical activity. Accordingly, the type of physical activity, in other words, a corresponding acceleration activity pattern, may be an important factor to consider when calculating the calorie consumption. As shown in illustration (c) of FIG. 2, although a user's heart rate may be normal, when the user is running, more calories are consumed than when the user is walking.

As described above with reference to FIG. 2, when calculating calorie consumption, according to an embodiment of the present invention, the calorie consumption and heart rate may be considered to have an identifiable relationship, e.g., a linear relation. A user's normal heart and activity pattern may be relevant as factors when calculating the calorie consumption. Accordingly, depending on the correlation therebetween, a calorie consumption calculating formula, according to an embodiment of the present invention, may be defined by the following Equation 1
Cal=a*(HR−HR₀)+Cal₀ Equation 1

Here, HR is a user's heart rate at a point in time when calorie consumption is to be calculated, and HR₀ is the user's normal heart rate, with Cal₀ being the user's normal calorie consumption. Namely, in the aforementioned graphs of FIG. 2, with respect to the correlation between the calorie consumption and heart rate, HR₀ can be a heart rate axis-intercept, Cal₀ can be a calorie consumption axis-intercept, and a can be a slope of the graph. Here, a may be determined based on the type of physical activity that a user is performing, which corresponds to the user's activity pattern.

Accordingly, to calculate the calorie consumption, according to an embodiment of the present invention, the user's normal heart rate and normal calorie consumption may have to be measured first, for example. Also, the user's heart rate may have to be measured at a point in time when the calorie consumption is to be calculated, and the user's activity pattern may have to be determined. Hereinafter, a calorie consumption calculating method will now be described in greater detail.

Referring again to FIG. 1, as described above, the memory 150 may maintain an activity pattern table, storing/recording at least one activity pattern identifier and a corresponding slope value for each said at least one activity pattern identifier. The activity pattern table will now be described in greater detail with reference to FIG. 3.

FIG. 3 is a table illustrating an example of an activity pattern table recorded in a memory according to an embodiment of the present invention.

As described above in FIG. 2, when calculating calorie consumption, according to an embodiment of the present invention, the calorie consumption may vary depending on the user's activity pattern. Further, the activity pattern may be taken into consideration through the slope a, in Equation 1. In FIG. 3, the slope a may be recorded as a slope value in an activity pattern table 300 of FIG. 3. In such an embodiment, the slope value may vary depending on each activity pattern. For example, when the activity pattern identifier is “walking”, the slope value may be a_{w}, and when the activity pattern identifier is “running”, the slope value may be aᵣ. A slope value, according to each activity pattern identifier may be determined via a predetermined test, such as a well-known multiple regression analysis. Also, the slope value may be determined via various methods, in accordance with arbitrary judgment of those skilled in the related art.

Referring again to FIG. 1, for storage of the activity pattern table 300, as above, the memory 150 may be configured into a memory including a Universal Serial Bus (USB) memory, a CompactFlash (CF) memory, a Secure Digital (SD) memory, a mini SD (miniSD) memory, an extreme digital (XD) memory, a Memory Stick, a Memory Stick Duo, a SmartMedia Card (SMC) memory, a Multi Media Card (MMC) memory, or a Reduced Size MMC (RS-MMC) memory, for example. Similarly, the memory 150 may be configured into a hard disk used in a general personal computer or notebook. The memory 150 may be an embedded type included in the calorie consumption calculating apparatus 100 or an external type. The memory 150 is further capable of supporting not only the aforementioned memory types but also all memory types which may be developed in the future, such as phase-change random access memory (PRAM), ferroelectric random access memory (FRAM), and magnetic random access memory (MRAM), noting that alternative embodiments are equally available

When the user's activity pattern is determined by the activity pattern determination unit 130, for example, and the slope a of Equation 1 has been determined, the heart rate sensor 120 may measure the user's heart rate, i.e., determine the HR value of Equation 1.

The user's normal heart rate and calorie consumption may be output to calculate the user's calorie consumption. The user's normal heart rate may further be determined as the heart rate usually measured before the user starts performing physical activity.

The user's normal calorie consumption may be also output by the data controller 140, with the normal calorie consumption being an amount of calories usually consumed when the user does not perform some physical activity.

The data controller 140 may perform an inductive method, for example, based on Equation 1, to calculate the user's normal calorie consumption. Namely, the normal calorie consumption of Equation 1 may be used as a basis for Equation 2
Cal₀=Cal−a*(HR−HR₀) Equation 2

Here, if it is assumed that the user is walking, for the physical activity, the slope value a, the heart rate HR while walking, and the user's normal heart rate HR₀ may be determined, and assigned as predetermined values. Accordingly, if calorie consumption according to walking of the user is output, the user's normal calorie consumption can be calculated.

Thus, the calorie consumption for walking may be output via the acceleration sensor 110, as in the conventional systems. Accordingly, FIG. 4 illustrates a correlation between the quantity of motion and calorie consumption, according to an embodiment of the present invention. In FIG. 4, the illustrated “+” marks are mapped for each calorie consumption, in accordance with the quantity of motion measured for each user. As illustrated in FIG. 4, the quantity of motion and calorie consumption may be considered to be in linear proportion to each other. As further shown in FIG. 4, when walking or running, if the different quantity of motions are different from each other, and increasing, the calorie consumption also increases in proportion to the quantity of motion.

Accordingly, the calorie consumption may be calculated by the following Equation 3

$$Cal = b * VM + c \\ VM = \sqrt{{\sum }_{t = x , y , z} \int {a}_{i} ⅆ t} \qquad Equation 3$$

Here, b and c may be constants determined based on the physical characteristics of a user, with aᵢ being an acceleration signal output from the acceleration sensor 110.

As described above, when the user performs walking, as the physical activity, the corresponding calorie consumption may be calculated via Equation 3 by using an acceleration signal output from the acceleration sensor 110. Also, the user's normal calorie consumption may be calculated by Equation 2.

The data controller 140 may output the user's normal calorie consumption through certain processes, such as Equations 2 and 3. The data controller 140 may further calculate the user's calorie consumption by performing calibration according to a calculation process of Equation 1, for example.

Namely, the data controller 140 may first determine a user's activity patterns, according to any type of physical activity that the user performs, and may later calculate the user's calorie consumption by using the user's heart rate. Accordingly, according to an embodiment of the present invention, the data controller 140 may measure substantial calorie consumption more accurately than a calculating of calorie consumption by using only an acceleration signal. Further, when calculating the calorie consumption, the data controller 140 may perform calibration reflecting a user's normal calorie consumption. Accordingly, through an embodiment of the present invention, it is possible to calculate calorie consumption based on the physical characteristics of each user. Also, when performing the calibration, the data controller 140 may calculate calorie consumption by using an acceleration sensor. Accordingly, according to an embodiment of the present invention, it is possible to embody a calorie consumption calculating apparatus using both an acceleration sensor and a heart rate sensor.

When the activity pattern determination unit 130 can not determine the user's motion from an acceleration signal output from the acceleration sensor 110, the data controller 140 may read the user's heart rate measured via the heart rate sensor 120. Here, when it is determined that the user's measured heart rate is less than a predetermined value, for example, the data controller 140 may determine the user is not performing any physical activity, or a sufficient physical activity, and calculate the user's calorie consumption as being zero, which allows the users to discern the actual calories consumed by their physical activities.

However, when the user's measured heart rate is more than such a predetermined value, the data controller 140 may amplify the acceleration signal output from the acceleration sensor 110. After this, the activity pattern determination unit 130 may determine the user's activity pattern by using the amplified acceleration signal. The activity pattern could be represent cycling, weight training, or the like, for example, which would be an activity pattern that the acceleration sensor 110 has difficulty in measuring a user's motion.

As described above, even when the physical activity, such as cycling or weight training, for which the acceleration sensor has difficulty in measuring a user motion, the calorie consumption calculating apparatus 100, according to an embodiment of the present invention, may determine that the user is performing the above physical activity by measuring the user's heart rate. Namely, the calorie consumption calculating apparatus 100 may calculate a user's calorie consumption more accurately regardless of the activity pattern of the user. In other words, the calorie consumption calculating apparatus 100, according to an embodiment of the present invention, may perform more accurate and effective calorie consumption calculations by effectively using both an acceleration sensor and a heart rate sensor.

According to an embodiment of the present invention, the data controller 140 may perform wired/wireless communication to transmit the calculated calorie consumption to an external communication terminal via the communication module 160, for example. The external communication terminal may include a watch 171, a mobile terminal 172, or a PC 173, for example, such as a notebook or the like, noting that alternative embodiments are equally available. The communication terminal may further provide the calculated calorie consumption to the user by receiving the transmitted calorie consumption and displaying the same on a predetermined display, for example.

To operate as described above, the communication module 160 may further include short-distance communication modules performing short-distance communication, such as wireless local area network (WLAN), Bluetooth, Ultra-wideband (UWB), Infrared Data Association (IrDA), Home Phone-line Networking Alliance (HPNA), Shared Wireless Access Protocol (SWAP), Institute of Electrical and Electronics Engineers standard 1394 (IEEE 1394), and the like, for example. In addition, the communication module 160 may support at least one of various access methods associated with any existing mobile communications such as Code Division Multiple Access (CDMA), Wideband CDMA (WCDMA), all IP, Global System for Mobile Communication (GSM), General Packet Radio Service (GPRS), not to mention a public switched telephone network (PSTN) access method, for example. The communication module 160 may be embodied to support at least one protocol of H.323, Media Gateway Control Protocol (MGCP), Session Initiation Protocol (SIP), and call control protocol for connecting a Voice over Internet Protocol (VoIP) call such as Megaco, for example. Alternative embodiments are equally available

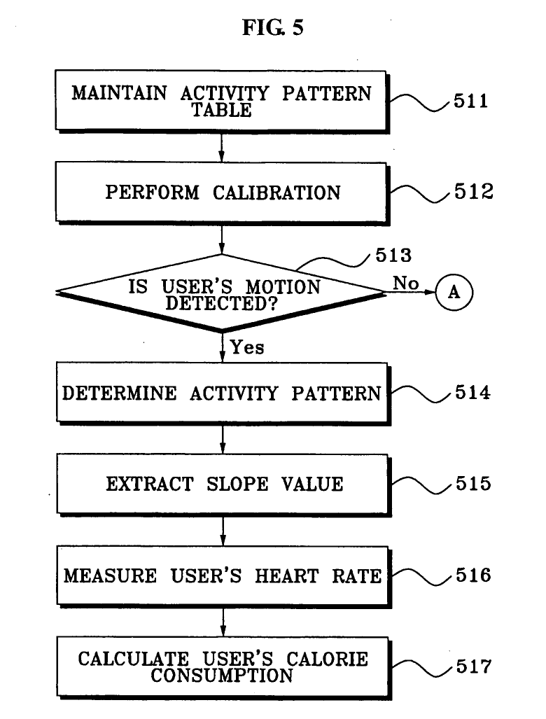

FIG. 5 illustrates a calorie consumption calculating method, according to an embodiment of the present invention.

In operation 511, an activity pattern table may be maintained, e.g., through a calorie consumption calculating apparatus. The activity pattern table stores and/or records at least one activity pattern identifier and a corresponding slope value, for example, for each of at least one activity pattern identifier. In operation 512, a calibration can be performed to measure a user's normal heart rate and normal calorie consumption when the user is not performing physical activity.

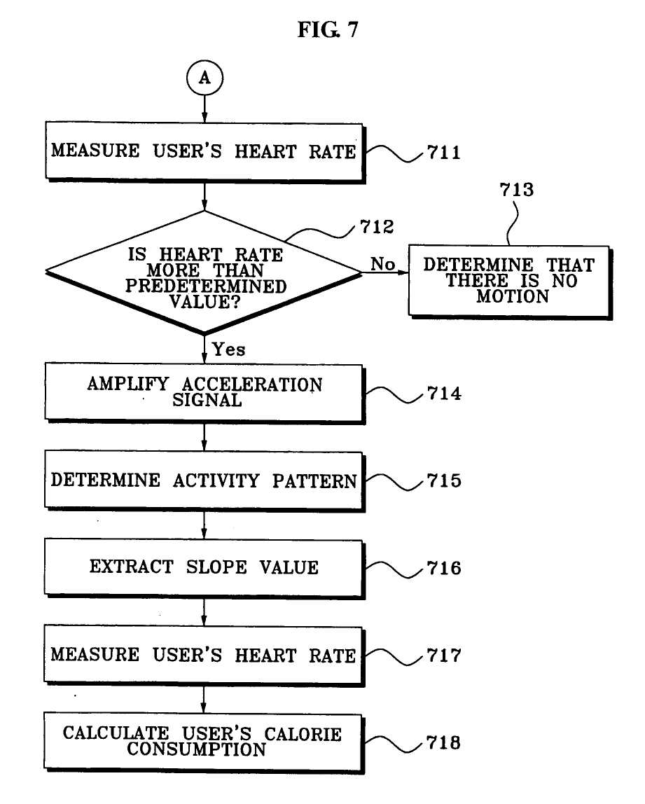

In operation 513, after performing the calibration, a determination of whether there is sufficient motion can be performed, e.g., via a predetermined acceleration sensor attached to the user. When the user's motion is not detected in the operation 513, further operations may be available, such as that described in detail in FIG. 7.

In operation 514, the user's activity pattern can be calculated according to the motion, when the user's motion is detected, in operation 513. In operation 515, a slope value corresponding to the determined activity pattern may be extracted when the user's activity pattern is determined.

In operation 516, the user's heart rate may be measured, e.g., via a predetermined heart rate sensor attached to the user. In operation 517, the user's calorie consumption may be calculated by using the extracted slope value and the measured heart rate. The method of calculating the calorie consumption in operation 517 may be similar to the calorie consumption calculating method embodiments of the calorie consumption calculating apparatus described in FIGS. 1 to 4, for example. Thus, a detailed description related thereto will be omitted.

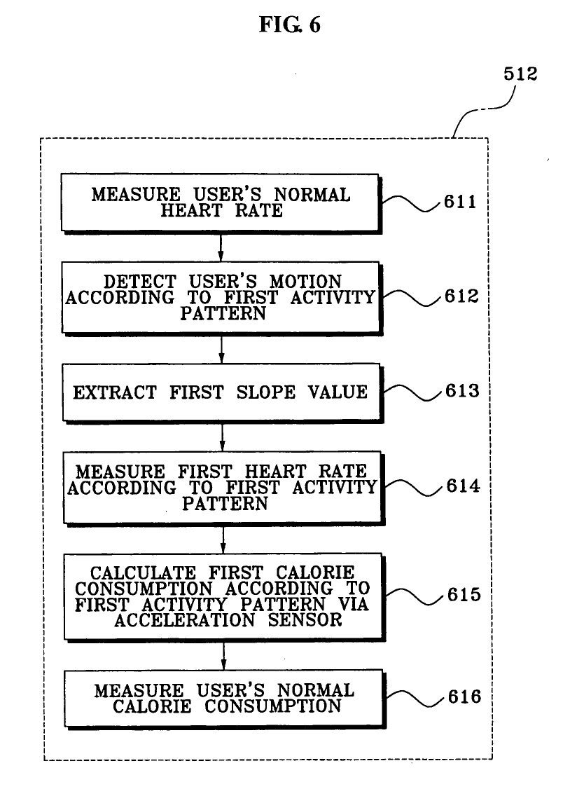

FIG. 6 illustrates a calibration method based of the characteristics of each user, according to an embodiment of the present invention.

A calorie consumption calculating apparatus, according to an embodiment of the present invention may perform such calibration based on the physical characteristic of each user, as illustrated in the operation 512 of FIG. 5. In operation 611, a user's normal heart rate may be measured. In operation 612, the user's motion may be detected according to a first activity pattern, for example.

In operation 613, a first slope value, corresponding to the first activity pattern, may be extracted from an activity pattern table. In operation 614, the user's first heart rate may be measured, while performing physical activity, corresponding to the first activity pattern. In operation 615, a first calorie consumption may be calculated according to the first activity pattern by using an acceleration signal output from the acceleration sensor. In operation 616, the user's normal calorie consumption may be calculated by using the first calorie consumption, normal heart rate, first slope value and first heart rate.

The method of calculating the normal calorie consumption in operation 616, according to an embodiment of the present invention, may be the same as a normal calorie consumption calculating method, e.g., for a calorie consumption calculating apparatus described in FIGS. 1 to 4. Thus, detailed description related thereto will be omitted.

FIG. 7 illustrates a calorie consumption calculating method for when an acceleration sensor, e.g., of a calorie consumption calculating apparatus, according to an embodiment of the present invention, does not detect a user's motion.

As described above, in operation 711, the user's heart rate may be measured when the acceleration sensor, e.g., of the calorie consumption calculating apparatus, in operation 513 of FIG. 5, fails to detect the user's motion.

When the heart rate is less than a predetermined value in operation 712, a calorie consumption calculating apparatus, for example, may determine that there is no motion of the user, and calculate the user's calorie consumption as zero, in operation 713.

When the heart rate is determined to be more than a predetermined value, in the operation 712, the acceleration signal output from the acceleration sensor may be amplified, in operation 714. Thereafter, in operation 715, the user's activity pattern may be determined based on the amplified acceleration signal. In the operation 715, the corresponding activity pattern may be determined, for example, to be cycling, weight training, or the like which are activity patterns the acceleration sensor would have difficulty in detecting motion.

In operation 716, a slope value, corresponding to the determined activity pattern, may be extracted from the activity pattern table, e.g., of a memory. In operation 717, the user's heart rate may be measured. In operation 718, the user's calorie consumption may be calculated. The method of calculating the calorie consumption in the operation 718, according to an embodiment of the present invention, may be the same as in a calorie consumption calculating method, e.g., for a calorie consumption calculating apparatus described in FIGS. 1 to 4. Thus, the detailed description related thereto will be omitted.

As described above, even with physical activity, such as cycling or weight training, for which an acceleration sensor has difficulty in measuring a user motion, a calorie consumption calculating apparatus according to an embodiment of the present invention may detect that the user performs the above difficult physical activity determination by measuring the user's heart rate. Namely, a calorie consumption calculating apparatus, according to an embodiment of the present invention, may calculate a user's calorie consumption more accurately regardless of the activity pattern of the user, by effectively using both an acceleration sensor and a heart rate sensor.

Embodiments of the present invention may further include a medium, e.g., a computer readable media, having computer readable code, e.g., program instructions to implement various operations embodied by a computer. The media may further include, alone or in combination with the computer readable code, data files, data structures, tables, and the like, for example. The media may include those specially designed and constructed for the purposes of embodiments of the present invention, for example, and/or they may include magnetic media such as hard disks, floppy disks, and magnetic tape, optical media such as CD ROM disks, magneto-optical media such as optical disks, as well as hardware devices that are specially configured to store and/or transfer and implement such computer readable code, such as read-only memory devices (ROM) and random access memory (RAM). The media may also be a transmission medium such as optical or metallic lines, wave guides, etc., including a carrier wave transmitting signals specifying the computer readable code, data structures, etc. Examples of such computer readable code may include both machine code, such as produced by a compiler, and files containing higher level code that may be executed by the computer using an interpreter, for example. The described hardware devices may be configured to act as one or more software modules, for example, in order to perform the operations of embodiments of the present invention.

According to a calorie consumption calculating apparatus, medium, and method embodiment of the present invention, it is possible to calculate calorie consumption of a user performing some physical activity by considering the user's heart rate and the type of physical activity, and accurately calculate calorie consumption by using both an acceleration sensor and a heart rate sensor.

In addition, according to a calorie consumption calculating apparatus, medium, and method embodiment of the present invention, it is possible to measure a user's normal heart rate and calorie consumption, reflecting the measured normal heart rate and calorie consumption on the calculation of calorie consumption, and thereby, for each user, calculate substantially accurate calorie consumption which may vary according to the physical characteristics of each user.

Further, according to a calorie consumption calculating apparatus, medium, and method embodiment of the present invention, it is possible to determine a user's activity pattern by measuring the user's heart rate, e.g., via a heart rate sensor, when the user's activity pattern is not detected by an acceleration sensor attached to the user, and calculate accurate calorie consumption of the user according to the corresponding activity pattern which is difficult to detect via the acceleration sensor alone, such as cycling or weight training.

Also, according to a calorie consumption calculating apparatus, medium, and method embodiment of the present invention, it is possible to embody U-HealthCare by transmitting, and potentially, displaying a user's calculated calorie consumption to an external communication terminal via a wired/wireless communication.

Although a few embodiments of the present invention have been shown and described, it would be appreciated by those skilled in the art that changes may be made in these embodiments without departing from the principles and spirit of the invention, the scope of which is defined in the claims and their equivalents.

What is claimed is:

1. A method for calculating calorie consumption of a body, comprising:
detecting whether the body is in motion based on an acceleration sensor for the body, and determining an activity pattern of the body according to a detected motion, if the motion is detected;
measuring the body's heart rate based on a heart rate sensor for the body; and
calculating the body's calorie consumption based on a predetermined relationship, between heart rates and the determined activity pattern, and the measured heart rate.

2. The method of claim 1, wherein the predetermined relationship is a slope value representing an identifiable change in heart rates for a particular activity pattern.

3. The method of claim 1, further comprising:
maintaining an activity pattern table recording at least one activity pattern identifier and a corresponding predetermined relationship, between heart rates and a respective activity pattern identifier, for activity pattern identifiers in the activity pattern table.

4. The method of claim 1, further comprising measuring the body's normal heart rate and normal calorie consumption, the normal heart rate and calorie consumption being measured when the body does not perform a predetermined physical activity,
wherein the calculating of the body's calorie consumption by using the predetermined relationship and the measured heart rate comprises multiplying the predetermined relationship by a subtraction of the normal heart rate from the measured heart rate and then adding the normal calorie consumption.

5. The method of claim 4, wherein the measuring of the body's normal calorie consumption comprises:
when a motion according to a first activity pattern is detected for the body, extracting a first predetermined relationship corresponding to the first activity pattern from an activity pattern table;
measuring the body's first heart rate according to the first activity pattern;
calculating the body's first calorie consumption according to the first activity pattern by using an acceleration signal output from the acceleration sensor; and
multiplying the first predetermined relationship by a subtraction of the normal heart rate from the first heart rate, and subtracting a result of the multiplication of the first predetermined relationship from the first calorie consumption.

6. The method of claim 1, further comprising:
measuring the body's heart rate via the heart rate sensor and calculating the calorie consumption during a physically motionless period, based on the body not being detected to be in motion by the acceleration sensor, based on the measured body's heart rate;
amplifying the body's acceleration signal, measured from the acceleration sensor, when the measured heart rate is more than a predetermined value; and
detecting the body's activity pattern based on the amplified acceleration signal.

7. The method of claim 1, further comprising:
transmitting the calculated calorie consumption to a predetermined communication terminal through a wired/wireless network,
wherein the communication terminal receives the calculated calorie consumption and displays the calculated calorie consumption on a predetermined display or the communication terminal receives the activity pattern and calculated calorie consumption with time data and displays the activity pattern and calculated calorie consumption with time data on a predetermined display.

8. The method of claim 1, further comprising:
calibrating potential predetermined relationships for corresponding activity patterns to reflect physical characteristics of the body by requiring the body to perform a predetermined testing process.

9. An apparatus, comprising:
an acceleration sensor for a body to detect motion of the body and to output an acceleration signal relative to a detected motion;
an activity pattern determination unit to determine the body's activity pattern based on the acceleration signal and a predetermined relationship, between heart rates and the determined activity pattern, corresponding to the determined activity pattern;
a heart rate sensor for the body to measure the body's heart rate; and
a data controller to calculate the body's calorie consumption based on the predetermined relationship and the measured heart rate.

10. The apparatus of claim 9, wherein the predetermined relationship is a slope value representing an identifiable change in heart rates for a particular activity pattern.

11. The apparatus of claim 9, further comprising a memory storing at least one activity pattern identifier and a corresponding predetermined relationship, between heart rates and a respective activity pattern identifier, for activity pattern identifiers in the activity pattern table.

12. The apparatus of claim 9, wherein:
the heart rate sensor further measures the body's normal heart rate, the normal heart rate being measured when the body does not perform a predetermined physical activity, and
the data controller further calculates the body's normal calorie consumption by using an acceleration signal of the acceleration sensor, the normal calorie consumption being measured when the body does not perform the predetermined physical activity, and calculates calorie consumption by multiplying the predetermined relationship by a subtraction of the normal heart rate from the measured heart rate and then adding the normal calorie consumption.

13. The apparatus of claim 12, wherein:
when a motion of the body for a first activity pattern is detected via the acceleration sensor, the activity pattern determination unit extracts a corresponding first predetermined relationship corresponding to the first activity pattern from an activity pattern table,
the heart rate sensor measures the body's first heart rate according to the first activity pattern, and
the data controller calculates the body's first calorie consumption according to the first activity pattern based on the acceleration signal output from the acceleration sensor, and calculates the body's normal calorie consumption by multiplying the first predetermined relationship by a subtraction of the normal heart rate from the first heart rate, and subtracting a result of the multiplication of the first predetermined relationship from the first calorie consumption.

14. The apparatus of claim 9, further comprising:
a communication module to transmit the calculated calorie consumption to an external communication terminal via a predetermined wired/wireless network.

15. At least one medium comprising computer readable code to implement the method of claim 1.
